(19) United States
(12) Patent Application Publication
(10) Pub. No.: US 2017/0035330 A1
(43) Pub. Date: Feb. 9, 2017

(54) Mobility Assessment Tool (MAT)

(71) Applicant: Frank E. Bunn, Thornhill (CA)

(72) Inventors: Frank E. Bunn, Thornhill (CA); Stacie Bunn, Markham (CA); Jingbo Zhao, Toronto (CA)

(73) Assignees: Stacie Bunn, Markham (CA); Jingbo Zhao, Toronto (CA)

(21) Appl. No.: 14/819,934

(22) Filed: Aug. 6, 2015

(51) Int. Cl. A61B 5/11; A61B 5/00; G06N 5/04; G06N 99/00; G06N 7/02

(52) U.S. Cl. CPC A61B 5/1128; G06N 99/005; G06N 7/02; G06N 5/04; A61B 5/112; A61B 5/1124; A61B 5/7267; A61B 5/0077; A61B 2576/00

(57) ABSTRACT

Revealed are the algorithms for a purely objective, reliable and reproducible mobility assessment tool (MAT) system with computerized fuzzy-logic algorithms that administer complex bio- mechanical assessments of a subject's static and dynamic balance and locomotion during performing 8 movements, by the administration of those algorithms to the skeleton nodal data stream representation of the movements derived from the 3-D video data stream observations or recordings of the subject performing the movements according to established kinesiology mobility standards. Each specific movement is measured as a function of 13 specific features' values that are determined by that function's algorithms for which there are adjustable parameters that allow calibrating the range limits of each feature's value of the assessments for select populations including gender, age, athleticism, and injury or disease subgroups.

### REFERENCES CITED

1. Tinetti, M. E., Williams, T. F. and Mayewski, R., “Fall risk index for elderly patients based on number of chronic disabilities,” American Journal of Medicine, vol. 80, no. 3, pp. 429-434, 1986.
2. U.S. Pat. No. 7,988,647 Aug. 02, 2011 Frank E. Bunn Class 600/595
3. U.S. Pat. No. 7,999,857 Aug. 16, 2011 Frank E. Bunn Class 348/211.1
4. USPTO Patent App. 20060190419 Aug. 24, 2006 Frank E. Bunn Class 706/2
5. USPTO Patent App. 20100049095 Feb. 25, 2010 Frank E. Bunn Class 600/595
6. USPTO Patent App. 20140024971 Jan. 23, 2014 Frank E. Bunn Class 600/595

### BACKGROUND OF THE INVENTION

The present invention relates to the algorithms of the Mobility Assessment Tool (MAT) systems and methods of determining and assessing the mobility of a subject through the administration of fuzzy logic computer vision algorithms to 3-D video derived multi-joint skeletal representation of the subjects' moving body. The MAT fuzzy logic computer machine learning performs administration of complex bio-mechanical assessments of the subject's static and dynamic balance and locomotion. The subject's kinematics are assessed and monitored to determine level of function against normative values. Algorithm parameters may be adjusted as required, based on new and established best practices, for select populations including gender, age, athleticism, and disease or injury subgroups.

The MAT is designed to save time for both administrators and health care professionals. Algorithmic assessments results provide both gross overall scores and detailed results of a subject's performance of movements. Provided in a readily accessible test format, results can quickly and easily be recorded within computer database frameworks based on kinesiology practice and protocol, and output in printable standardized kinesiology report formats of the numerical and textural mobility assessment results and recommendations.

### SUMMARY OF THE INVENTION

In general terms, the present invention provides a system, the Mobility Assessment Tool (MAT), for assessing the mobility of a subject, said system comprising: two or more motion sensors to observe movement of a subject performing 8 simple movements and to generate and record a 3-D video digital data stream representative of such movements; an active logic engine administering computer vision technologies including machine vision and machine learning functions to determine and record from the video a multi-nodal skeleton representation of 20 to 25 physical joints of a subjects' body movement for each video frame of the moving subject which representation is isolated from the stationary background; a set of fuzzy logic computerized active logic engine machine vision algorithms each with one or more adjustable parameters that can be administered for interpreting the kinesiology defined kinematics of each movement within which the movement can be determined by the said engine administering machine learning logic, as the measure of level of function of the movement lying within or lying outside of normative range of values of 13 specific features' values of the movements; and the administration of the algorithms these features' values by which to determine the mobility assessment of the subject; and automated output of assessment results as a readily accessible text format for standardized reports in numerical and text interpretations of the assessment.

Through computerized automation the MAT provides consistent, reliable and reproducible mobility assessment results across testers administering the MAT tests of a subject's mobility. Every subject receives identical computerized verbal and video instructions each time they perform the assessment test thereby eliminating tester and intra-tester reliability as a source of error. Verbal instructions can be provided in a variety of languages to suit the subject being assessed.

The MAT is designed to save time for both administrators and health care professional. Assessment results provide both gross overall mobility scores and detailed results of the subject's performance of test movements. Provided in a readily accessible text and numerical formats, the results can be recorded within internal system frameworks, based on practice setting. These formats provide to mobility practitioners, automation efficiency reducing their time to make and report a subject's mobility assessment while increasing the consistency of those assessments.

The MAT tool provides to mobility practitioners, a computer-automated, objective, reproducible and reliable assessment in keeping with kinesiology standards of measurement of the mobility of a subject and potential related medical conditions and remedial procedures, providing the critical information the practitioner needs for diagnosis of the subject's condition, injury, illness, or affliction and the treatments that may be needed.

In a further aspect, the invention provides a method of assessing mobility of a subject comprising the steps of recording motion of said subject, administering fuzzy logic machine vision and machine learning algorithms with said active logic engine, on said motions to determine kinematic assessments of mobility and for determination of abnormalities of such movement, determining relationships of said abnormalities to known normative values, and determining whether said abnormalities are within a known norm or range of known norms.

In a further aspect, the invention provides methods and systems of administering an allocator on said active logic engine to determine if said abnormalities are within a known normative value or range of known normative values whereby to determine the possible existence of bio mechanical or neurological conditions or injuries of the subject and to determine at what stage are the said conditions or injuries. The invention further can infer from these determined conditions or injuries what are their relationships to known kinesiology rehabilitation procedures and treatments for such conditions or injuries, and can determine the potentially appropriate rehabilitation procedures recommended by the said methods and systems to relieve, repair or restore the subject's health and potentially cure the said conditions or injuries.

From the above, it will be clear the determination of mobility impairment will include the deterioration of the walking gait of a subject. It has been shown by extensive studies that the deterioration in mobility, including gait, of a subject has been directly correlated to neurological deterioration of the subject. Dr. Dean M. Wingerchuk at the Mayo Clinic in Rochester Minn. has reported “Gait analysis adds objective, reliable outcome measures sensitive to detecting neurological deterioration.” Dr. Wingerchuk states that “Gradual deterioration in ambulatory function is one of the major manifestations of progressive forms of Multiple Sclerosis”. At the Alzheimer's Association International Conference 2012 in Vancouver, Canada, three independent research studies each surveying more than 1,000 people, all confirmed mobility deterioration in gait of subjects directly reflected their neurological deterioration due to their Alzheimer's dementia. The studies were conducted by Dr. Stephanie A. Bridgenbaugh of the Basel Mobility Center in Basel, Switzerland; Dr. Mohammad Ikram at Erasmus MC Rotterdam, the Netherlands; and Dr. Rodolfo Savica of the Mayo Clinic Study of Aging, Rochester Minn.

From the above, it will be clear the assessment methods and system means described could be applied to the determination of mobility impairment including the deterioration of the walking gait of a subject to determine the potential existence of brain related illnesses including but not limited to Multiple Sclerosis and Alzheimer's dementia.

In this example, the expert system administers the active logic engine algorithms to the data available to identify that a mobility impairment condition exists in one or more movements in the current assessment and accesses a data base to determine relationships of this mobility impairment condition to a previous assessment for this subject, stored in the database component of this system, to determine if this mobility impairment condition was detected in a previous assessment. If the mobility impairment condition did so exist, the computer system, administering time derivative determinations, calculates the rate of change in the mobility impairment condition between successive assessments for this subject. The computer facility, using a predetermined baseline matrix of outcomes, then determines if a critical mobility impairment condition exists and, comparing to previous assessments, determines if a deterioration in the mobility impairment condition has occurred, and if so occurring computes the rate of change of this deterioration. This active logic engine algorithm function of the computer system can apply equally to the brain concussions and conditions of the subject as is discussed herein.

### BRIEF DESCRIPTION OF THE DRAWINGS

Embodiments of the invention will now be described by way of example only with reference to the accompanying drawings in which:

FIG. 1 is a schematic of a 3-D representation of a dual camera observation of the sit-stand-sit movement mobility assessment of a subject.

FIG. 2 is a schematic 3-D representation of a dual camera observation of the turn-in-a-circle, turn-on-the-spot and stumbling movements mobility assessment of a subject.

FIG. 3 is a schematic representation of a wobble movement functional assessment process for a wobble forwards, backwards, or possibly side to side.

FIG. 4 is a schematic representation of wandering deviation from normal movement mobility assessment of a subject illustrating wander from kinesiology walking standards for a straight normal path (4a) and for wander from normal foot-to-foot separation (4b).

FIG. 5 is a 3-D plot of left and right foot measurements that are made by the Mobility Assessment System with definitions of the features measured in the gait assessments.

FIG. 6 is a spreadsheet example of all the mobility assessment features measured for fourteen selected athlete subjects during the Walk and during the Turn-360 Degrees movements.

FIG. 7 is a spreadsheet example of the Thresholds established for each of the mobility assessment features measured for the Walk and the Turn-360 Degrees movements.

### DETAILED DESCRIPTION OF THE INVENTION

Prior to describing the system and its function in assessing mobility of a subject from observing the subject performing 8 specific movements: sit still in a chair; arise from a chair; stand still; stand still with eyes closed; walk in a straight line path; turn 360 degrees walking in a circle; and turn 360 degrees walking on-the-spot. A number of the typical assessment environments will be described to provide context to the operation of the system.

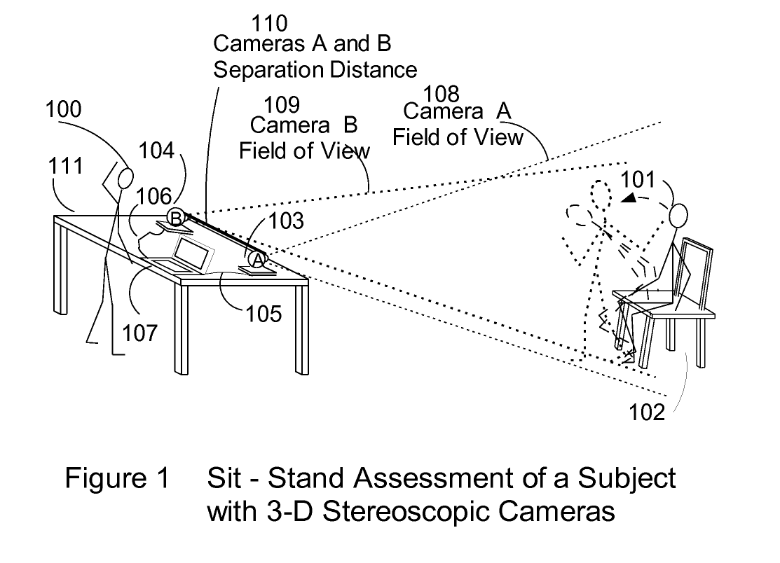

Referring to FIG. 1, an expert system apparatus is used within a typical professional office environment for observing and video-recording specific movements of a subject, (101). The system includes a computer (107) that implements an active logic neural networks decision engine, to administer the active logic engine algorithms to video data obtained from motion sensors (103 and 104). The motion sensors may be a camera or cameras operating in one or more of the visible, or infrared, or ultraviolet spectrum, an acoustic image capturing device or location sensors such as GPS positioning devices or RF motion/location devices from which to generate and record information of the movement of the subject. For convenience they will be collectively referred to as cameras. The expert system embedded in the computer (107) operates on and administers active logic machine vision engine algorithms to the video data stream from the cameras (103 &104) to derive and record a multi-joint skeleton nodal data stream with each node representation of one of the joints of the subject' body in each frame of the video. Administration to the skeleton representation, of additional active logic engine machine learning algorithms and tests enable the expert system to determine and record 13 specific features'’ values measured, to be described later, of the subjects' movement and determines whether the movements observed are an abnormal condition, that is, one that departs from expected or desired kinesiology standards of motion and commonly referred to as normative or normal motion. The system utilises that condition information to assess a particular condition, such as presence of a bio mechanical injury or neurological injury causing the limited level of compliance to the kinesiology standards for that movement.

In FIG. 1, a subject (101, solid lines) sitting in a chair (102) is being observed by a cameras (103 & 104), connected via wired or wireless interfaces (105 & 106) to the computer (107) being operated by a test facilitator (100). The test procedure conducted provide computerized voice instructions to the subject, requiring the subject to sit upright, straight and steady in the chair and the administration of the additional active logic engine algorithms to the derived the skeleton nodal data stream from which 13 specific features'’ values of the movements are determined as representation of the movement of the subjects' body and further administration by the engine of mobility algorithms to the set of feature values will detect any abnormal position or movement of each body joint of the subject represented by a node. The subject is then requested by the computerized voice to arise and the administration of the additional active logic engine algorithms to the skeleton nodal data stream and derived specific features'’ values representative of the rising movement of the subjects' body will detect any abnormal motion of that movement of subject arising from the chair from the measuring the 13 specific features' values in a process that will be detailed later herein. The cameras (103 & 104) detect the motion of the subject (101) and the expert system transfers and records the data representing the motion to the computer (107) for further processing.

As an example, say the subject takes two attempts to rise from the chair (101, dotted lines). The cameras (103 &104) capture the movement of the subject (101) in a time dependant manner and the data are transferred to the computer (107). As will be described more fully below, the expert system administers the new and uniquely developed mobility assessment movement algorithms being revealed herein, which will be referred to as Mobility Algorithms to determine normality or abnormality of the movement according to kinesiology standards of movement as derived from the 13 specific features' and applies this information and additional input to provide the criteria required to apply standardized kinesiology test criteria and test parameters. In the example provided, the two attempts to rise are determined as an abnormal mobility condition and these determinations indicate that the subject has a significant limited level of compliance to the kinesiology standards for that movement and defining the subject's impairment condition for that movement.

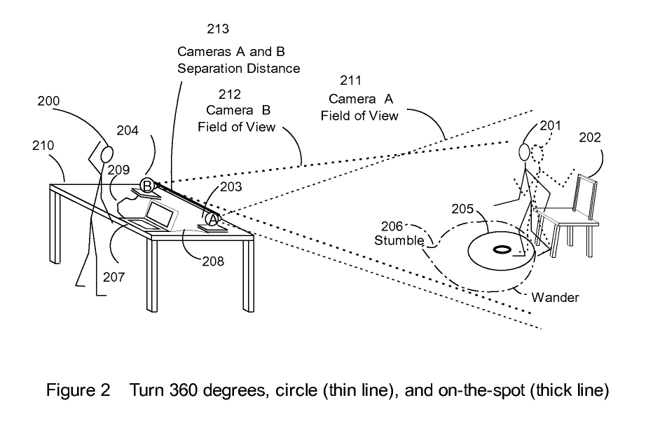

FIG. 2 shows a typical functional test assessment process and decision computations for a subject (201) having risen from a chair (202), to stand still, then turn around 360 degrees. The test facilitator (200) and the computerized voice instructions asks the subject, to stand still for assessing steadiness without wobbling or swaying. The computer (207) and the cameras (203 & 204) capture and record the video data of the movement indicated at (201), where solid lines stick-person subject and dotted lines stick-person subject indicate change of position over time to indicate that the subject is wobbling. In this example, the expert system, administering the algorithms in real time or to the recorded data, may determine the wobble or swaying as being a limited level of compliance to the kinesiology standards of mobility for that movement. These determinations are provided to the selected established kinesiology standards for those movement test procedures and mobility scoring, and, depending on the cumulative results, the expert system may decide the subject has a significant level of difficulty performing that movement (201). The expert system also determines the level of mobility of the subject's actions while standing (201), wherein wobbling, swaying or stumbling is detected, recorded and scored.

Continuing with this FIG. 2 example, the computerized voice instructions then asks the subject to turn 360 degrees in a circle along the path (205), for which the solid line indicates the expected circular track for normal turning. The expert system observes the actual movement (206) indicated by the dotted line and administers active logic engine algorithms to the sensor data to determine the wandering and stumbling as being a limited level of compliance to the kinesiology standards for that movement. This is input into established test procedures and mobility scoring to determine if the subject has significant mobility limited level of compliance to the kinesiology standards for that movement for that movement.

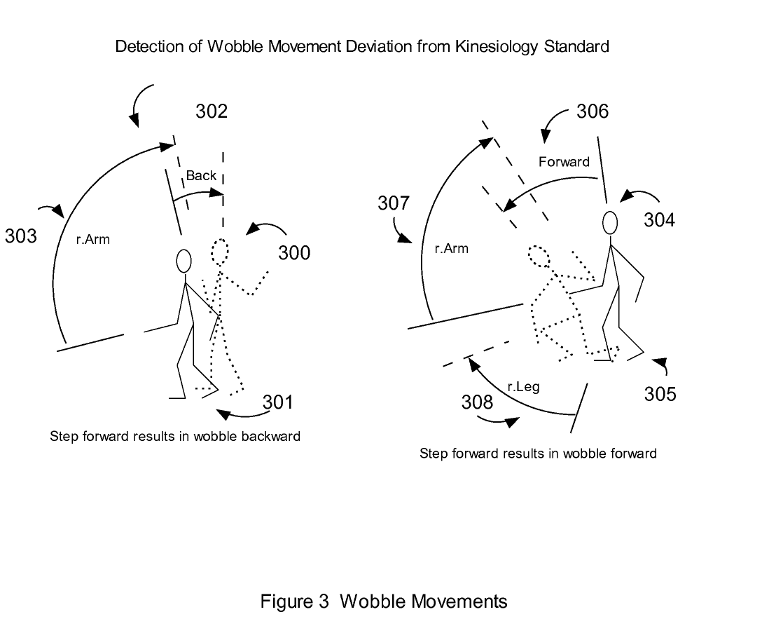

Eight kinesiology accepted movements have been selected that are used to observe and assess mobility, the occurrence of mobility impairment and conditions of a subject and the subject's potential of having related injury, illness, pain or disease for that subject being assessed. FIG. 3 illustrates examples of two movements of a subject which would normally be determined by a mobility assessment algorithm to deviate from expected normal or standard movement. Normal for a specific subject means movement that has been previously observed and recorded in databases for this subject and is accepted as a base level of compliance to the kinesiology standards for that movement. Standards for that movement can be defined as movements that have been observed and recorded in databases of typical movements for subjects of similar age, sex, health, and mobility and is accepted as a base level of compliance to the kinesiology standards for that movement for any similar subject.

Algorithms revealed in this invention are administered by the active logic engine algorithmic expert system, to the input video data streams from a multiplicity cameras to derive the skeleton nodal data streams and to derived specific features' values data streams, said additional algorithms functioning as an administrator, to conduct detection determinations, and specific features' extraction from the nodal data stream administrations, from which to assess the likelihood of limited level of compliance to the kinesiology standards for that movement for a subject. This is accomplished by administering active logic engine algorithms to video data, to develop for each frame of the video data stream a computerized frame by frame skeleton nodal data stream representation of the subjects' body including multiple control joints such as: head, neck, shoulders, elbows, wrists, hands, torso, hips, knees, ankles and feet. Further algorithms are administered to each skeleton nodal representation for each frame to determine a measurement of specific features' values of the movements of each joint relative to their location in the previous frame. Additional algorithms are administered to each measurement to determine metric amount of that joint's movement where by the algorithms can determine the bio mechanical movement of the subject's body at each joint. For example specific features' movements values such as for feet movements: step length, height of moving foot off the floor, separation between feet, step frequency can be determined. Another example for arm specific features'’ movements relative to: shoulder, elbow, wrist and torso joints, the angle of the upper arm and lower arm relative to the position of the torso can be determined from the angles formed by the wrist-elbow- shoulder joints. Not all such movement examples will be discussed here but it will be clear to any one informed in bio mechanics that with sufficient control joints, most bio mechanical body movements can be determined.

These specific features' values as determined by administration of the algorithms described above, also produce electronic or mathematical signatures of said movements such that administration of additional algorithms can derive from these movements, an allocator value to determine whether the values of said signatures are within known norms of the movement of personal, and/or, normal range level of compliance to the kinesiology standards for that movement and deviations there from for features'’ movement of normal subjects which provide features'’ signatures of movement are stored in the system in related databases. Then, deriving similar signatures of subjects to be assessed as to mobility performance of the movements, the active logic engine algorithms determine the deviation of these signatures from the normal signatures to make the decisions as to infer limited level of compliance to the kinesiology standards for that movement. If limited level is interpreted, the algorithms then determine whether the movement indicates a bio mechanical or neurological injury, pain, or illness and if so indicated, it informs the appropriate health care personnel or systems. Similarly, determinations of the deviation of subject's movements could result from medical emergencies such as heart attack, or seizure that such emergencies also require healthcare personnel assessment in responding to the subject in question for which appropriate medical actions can be taken.

The administration of the algorithmic system using the active logic engine, can implement unique determinations and subsequent reporting assessment results for mobility level of compliance to the kinesiology standards for that movement. These reports can be in readily accessible text format that can be cut and pasted into internal and external standardized reports based on kinesiology practice. Later, such observations of the subject will determine the changes in the subject's movement as it correlates to their earlier determinations and in real time determine any deviations that could relate to mobility reduced level of compliance to the kinesiology standards for that movement and possible existence of injury, pain or medical health condition as determined by the active logic engine algorithms. However, if the algorithm administration system through access to related databases has access to medical and health information and database of related mobility impairment signatures of the subject, the active logic engine processor may be able to determine if the subject being observed is in fact having a health problem such as heart attack, stroke, diabetic coma, epileptic seizure or brain related diseases such as Multiple Sclerosis, Parkinson's, Dementia, Cerebral Palsy, or brain concussion, and any of which could be needing immediate medical assistance and if so determined, can inform the proper health care providers.

In the case for that a subject is determined to have a reduced level of compliance to the kinesiology standards for a movement, for example as a stagger back shown in FIG. 3, the subject in attempting to step forward (solid line stick figure), actually staggers backward (dashed line stick figure) in which the major motions of the subject's back and right arm could be determined by the mobility impairment assessment algorithms administered to the specific features' values data stream, to have deviated from expected for either the normal or standard movement. Similarly a stagger from side to side could indicate impairment. In the stagger forward example, the subject in attempting to step forward (solid line stick figure), actually staggers forward (dashed line stick figure) in which the major motions of the subject's back and right arm and left leg could be determined similarly by the mobility impairment algorithm to deviate from expected for either the normal or standard movement. Details will be discussed later.

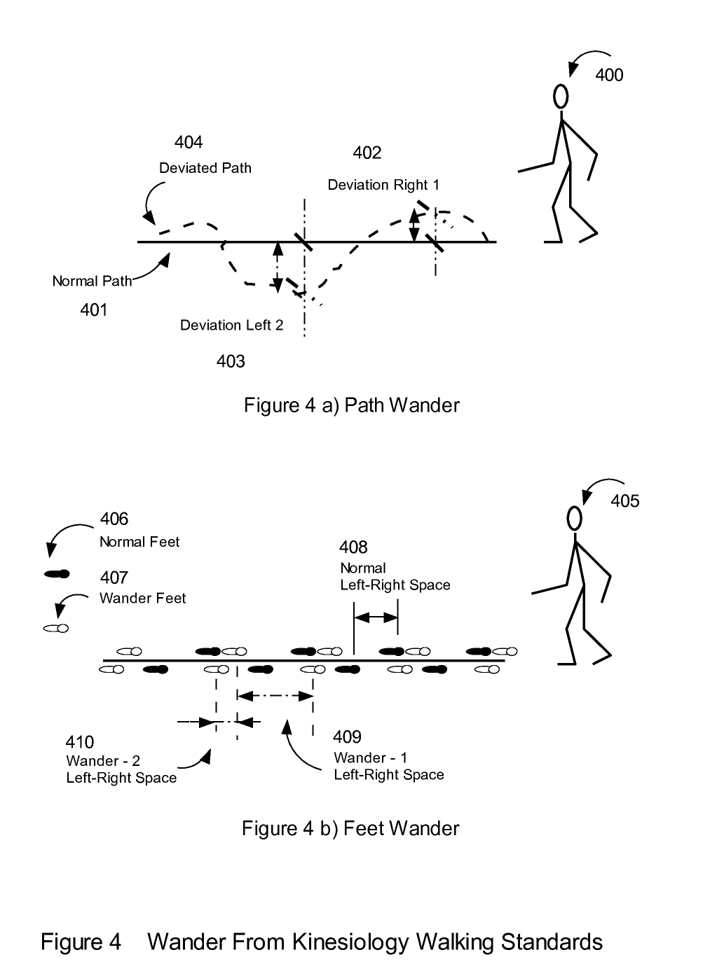

FIG. 4a) illustrates movements of (400) a subject's feet, normal (406) or wander (407), in which the subject's walking path wanders from a normal or standard path (401) for the subject's feet indicated by a Deviation Right 1 (402) and a Deviation Left 2 (403) which would be determined by the administration of walking algorithms to the skeleton nodal data stream of control joint data to deviate from expected for either the normal or standard movement. Further, FIG. 4b) illustrates specific features' movements derived from the skeleton nodal data stream of (405) a subject's feet which wander from the expected normal (408) or standard foot spacing where the subject's left to right Wander-1 (409) spacing is larger than expected and right to left Wander-2 (410) spacing is shorter than expected. The unexpected movements could be determined by the mobility level of compliance to the kinesiology standard movement's algorithms to deviate from expected for either the normal or standard bio mechanical movement. Details will be discussed later.

Further, a significant foot placement specific features' test while walking is to request the subject to walk toe-to-heal such that the subject places each foot at each step so that the heal of the front foot touches the toe of the back foot. This is a more difficult and perhaps stressful walking task for the subject and the mobility assessment of the subject's movement can determine more subtle effects of and existence of bio mechanical or neurological problem. Further, an even more difficult walking task is to request the subject to walk either regular walk or toe-to-heal walk but with the moving foot to cross over the stationary foot such that the subject's feet when both are stationary are crossed at every step in the walk. Mobility assessment of the subject, under the stress in this task, can determine even more subtle effects of and existence of bio mechanical or neurological problems. It will be obvious to anyone verse in bio mechanics, that many more movements will be applicable for administration of the algorithms revealed herein for mobility assessment, however for brevity are not detailed here.

The above examples relate to an assessment performed in a controlled environment by a medical practitioner, tester or operator. The MAT system incorporates computerized voice instructions for each movement the subject is requested to perform thereby providing consistent reproducible test procedures. The expert system may also be used in a normal non-clinical environment as a continuous, non-invasive mobility assessment tool, such as a mobile computer and cameras system implemented near an athletic playing field to provide quick on-sight assessment of athletes before, during or after play. Particularly if a player is suspected of having suffered a hit, shaking or injury to the body during play, a prompt mobility assessment at the time of such occurrence could be critical in assessment for potential bio mechanical or neurological problem and the expert system algorithms could be administered to alter health providers and practitioners such that immediate action for medical attention can be taken as needed.

The implementation of the expert system can be considered as having two main linked components: a basic mobility assessment system and an advanced mobility assessment system. The basic system permits an operator to control part or all of the assessment process and to input assessments of the mobility of the subject being assessed. The advanced system contains the algorithms and computer facility active logic engine neural networks decision computations with which the expert system determines the assessment outcomes and recommendations according to established parameters, the mobility assessment total score number, and the differential determination of current assessment to previous assessments, and generates reports of remedial actions, possible aids and healthcare procedures, to the subject, or to the subject's employers or to the caregivers of the subject.

Further, the expert system may be administered using a limited number of skeleton nodal control points such as head, shoulders, trunk, elbows, wrists, hands for monitoring larger arm movements. Alternatively the expert system could also use a larger number of control points including the above plus thumbs, fingers, knuckles for refined higher resolution of movements such as for observing shaking of hands that could be typical of diseases such as Parkinson's.

The advanced system can compute a larger number of skeleton nodal control points and related selected specific features' values assessed than does the basic assessment system, for each video frame. Using known video skeleton nodal control point to create additional points, the advanced system can then derive additional specific extracted features' with which to detect the finer more precise subject's movement of each control point from frame to frame based on the displacement of each control point on a given frame relative to the same control point on the previous frame by differentiating between those two to determine the control points that are moving and those that are stationary on a frame to frame basis.

This may be performed by determining a specific extracted feature such as whether a given skeleton nodal control point, E for example of an elbow movement, in the image frame, x, moves or is displaced by or more than say 3 video pixel spaces in any direction for this control point in its location in the next image frame, y. If so then this control point, E, in frame x is identified as moved and assigned pixel component location. If pixel E in frame x, moved less than 3 pixel spaces at its new location in frame y, then this control point, E, is identified as not moved and assigned the pixel components it had in frame x. By computing the movement of all control points from frame x to their locations in frame y and assigning all those that move 3 or more spaces, with the new pixel locations where they appear in frame y and all those control points in frame x that move less than 3 pixel spaces to retain their pixel locations from frame x, a skeleton motion-rendition of the subject's movements wherein all movement of the subject can be observed and movement assessed. The number of pixels, for example here being 3 or more, is set by an adjustable algorithm pixel parameter by which the administration of the pixel movement algorithms determines the number of pixels moved. Additionally, administration of the pixel movement algorithms to the 3-D data stream components of the cameras can determine the physical distance of the skeleton nodal control joint movement from frame to frame where the distance of the movement is set by an adjustable algorithm distance parameter input to the administration of the algorithms.

The finer movement and measurements resulting from the higher number of skeleton nodal control points can be considered as a higher resolution detection skeleton nodal data stream and derived specific features' values representation which in this case is the subject being mobility level of compliance to the kinesiology standards for that movement assessed, and stores that skeleton nodal data steam and derived features' values representational data in a database. It is preferred that the mobility impairment detection algorithms revealed herein are advances on and entirely new derivations of those stagger algorithms of U.S. Pat. No. 7,988,647, and U.S. Pat. No. 7,999,857, and networking algorithms of U.S. patent application 20060190419 and determination of medical conditions by measuring mobility patent application 20100049095, and assessment and cure of brain concussion and medical conditions by determining mobility patent application 20140024971, the contents of which are incorporated herein by reference.

By using such techniques, it is possible to evaluate if a particular movement is indicative of a mobility level of compliance to the kinesiology standards for that movement and if an impairment condition exists from determining the movements of a subject. Each of these evaluations may be made from the specific extracted features' values derived from the skeleton nodal data stream of the motion by determining the average deviation of a set of specific features' data representing the body, for example determining the average location of the centreline of the subject relative to the normal path for that movement.

The mobility assessment algorithms are administered to the real-time or recorded video data stream and to the associated skeleton nodal data for determination of the specific features' values measure of the movements by a subject according to the Tinetti mobility test requirements which are defined and accepted as following kinesiology standards and protocol. The eight selected movements of the subject are: sit still in a chair, arise from sitting in a chair, stand still, stand still with eyes closed, sit down on a chair, walk in a straight path, turn 360 degrees walking in a circle and turn 360 degrees turning on-the-spot. With these eight, simple movements, the administration of the mobility assessment algorithms of the MAT extracts 13 specific features' values measure of mobility parameters with which the mobility assessment determines if the measured numerical values of these features' are within the range of the thresholds set for each feature. Feature values lying outside these thresholds allow additional algorithms to determine the mobility abnormalities these out-of-range features' and may further determine the possible conditions, illness, injury, pain, disease of the subject indicative of such abnormalities.

In an alternative embodiment images from multiple cameras may be used as shown schematically in FIG. 1 (camera A 103 and camera B 104). One of these cameras could be an infrared illumination source and receiving detector and the other could be a visible detector such the Microsoft Kinect duel camera system utilized in the Microsoft games console. Both the original Kinect V-1 and the newer version Kinect V-2 have been implemented in this Mobility Assessment Tool (MAT) system and each has been found to be an inexpensive 2-camera sensor system with the added advantage of significantly improving separation of the background from the moving image of the subject. The data are composed into a stereoscopic 3-dimensional (3-D) representation of the subject's movements using known image reconstruction techniques, and the Kinect cameras can transform the images of the subject in the video recording to become an isolation of the moving subject with full retention of all movements of all of the subject's body including feet, legs, trunk, arms, hands and head while rendering the recording devoid of the information needed to identify the subject. Additionally, the Kinect video camera system has imbedded software that produces multiple skeleton nodes (20 for the V-1 and 25 for the V-2) which we have incorporated into the MAT assessment algorithms for making the measurements on the movement of these nodes video frame by video frame observations of a subject performing the movements of the Tinetti test. The MAT omits the Tinetti nudged a subject sub-assessment as herein it is considered to be an invasive interference of the subject.

For data acquisition, the Kinect sensor samples at a frequency of approximately 30 Hz and video frames are captured both in color and depth. Using captured frames, the middleware of Kinect software SDK, segments and tracks human skeletons and gives the output of a human skeleton represented by 20 nodes, for the Kinect V-1 and 25 for the V-2, or control points in the Kinect's own reference frame known as the skeleton space. Each node represents a specific joint with 3D position information in units of meters. The skeleton space uses a right-handed coordinate system: the Y axis lies in vertical direction of the image plane, the Z axis extends in depth perpendicularly from the sensor and the X axis is horizontal in the image plane and orthogonal to the Y and Z axes.

In pre-processing, the position and the speed of each joint in the time sequence are considered as one-dimensional signals. Two 2^{nd} order low-pass Butterworth smoothing filters were used to reduce the noise in the signals. Empirically-determined cut-off frequencies of 4 Hz and 1 Hz were used for the position and speed signals of each joint, respectively.

To extract the features' of walking steps, it is necessary to accurately segment the steps, i.e. determine the start and the end of a step. The Z component (in depth) of foot speed is used because it showed good regularity in relation to the phases of the steps. The algorithm robustly segments the steps while ignoring the small peaks generated by the interference from parts of the body overlapping or the distance between the subject and the camera being too long.

Algorithms determine the time series of the Z speeds of both feet during stepping. The most important features' are the start-, the mid- and the end-points. These are identified as feature points and used for analyzing the gait. The algorithm is insensitive to the tilt angle of the Kinect sensor since we use the Z component (in depth) of foot speed for step segmentation.

Algorithms finding overlaps of the feet in the 360° Turn analysis uses the same pre-processing step as the gait algorithms. Since a subject is turning 360° on spot, it is difficult to segment the steps using the method for the gait analysis. To measure the continuity of a turn, the algorithm identifies the skeleton frames in which the speeds of both feet are below a certain speed threshold. Specifically, the speed is defined as the Euclidean norm of X and Z components of the speed of a foot. A group of consecutive skeleton frames below a certain speed threshold indicates that a subject may have paused during a 360° turn. The algorithms identify pauses during the 360° turn based on a toe-off speed threshold of 0.2 m/s. The time interval of each pause is determined by the difference in timestamp of the first skeleton frame and the last skeleton frame in a group.

Several trunk features' are measured. The stability of the trunk of the body is monitored by two factors: the use of arms for balancing and the lean angle of the trunk in the coronal plane. Additionally, for gait algorithms it is necessary to calculate the deviation of the base of the spine relative to the traveled path.

It is assumed that at the start of an assessment the subject is not using the arms for balancing and the wrists are placed at the sides of body as directed by the computerized voice instructions. In other words, the wrists are at their resting positions. The distance between wrists is defined as the Euclidean norm of X and Y components of positions of two wrists. The Z component is ignored since the arms typically swing during walking.

During a walk or a 360° turn, when subjects use their arms for balancing or lean the trunk of their body, the distance between the wrists will increase. By calculating the difference of the distance between the wrists at the resting positions and the distance of wrists during a walk or a 360° turn, the use of arms for balancing can be detected. To illustrate the process, the algorithms detect the changes of X distances of two wrists with respect to the origin of the Kinect to measure the interval in which a subject may use an arm for balancing.

The leaning angle of the trunk is defined as the angle between the vector of the trunk (between the center of shoulders and the spine base) and the gravitational vector in the coronal plane. The angle can be obtained by calculating the mathematical dot product of these two vectors.

To measure the deviation from the path during a walk, a path vector P is calculated using the position of spine base in the first frame and last frame in a walk. The instantaneous deviation from the path is defined as the perpendicular distance between the position of the base of the spine and the straight line path along vector P.

There are several specific features' values extracted from the above measurements that further administration of algorithms will determine for the Walk Gait assessments. For the gait algorithms, of interest are the three feature points of each step: the start, the mid and the end. Each feature point contains the timestamp, the position and the speed of the moving foot. The gait specific features' values involved in the gait algorithms are the following (with units in parentheses):
1. Initiation of gait t 1 (in milliseconds): time consumed between computerized voice instruction “begin” and start of a walk by a subject;
2. Step Through Length for right foot: lr (in meters—left); (also for left foot: ll in meters):
mean distance between the ankles of two feet when both of them touch the ground during a walk;
3. Step Height for right foot: Sr (in meters per second); (also for left foot Sl: meters per second):
mean speed of a moving foot in the vertical direction;
4. Step Length for left foot: dl (in meters): length of left foot step from step-start at heel lift-up to step-stop at heel put-down;
5. Step Length dr (in meters): length of right foot step from step-start at heel lift-up to step-stop at heel put-down;
6. Step stance df (in meters): distance between the left foot heel at heel-down and right foot heel at heel-down;
7. Step Interval t2 (in milliseconds): time consumed between end of a right (or left) foot step and a start of new left (or right) foot step;

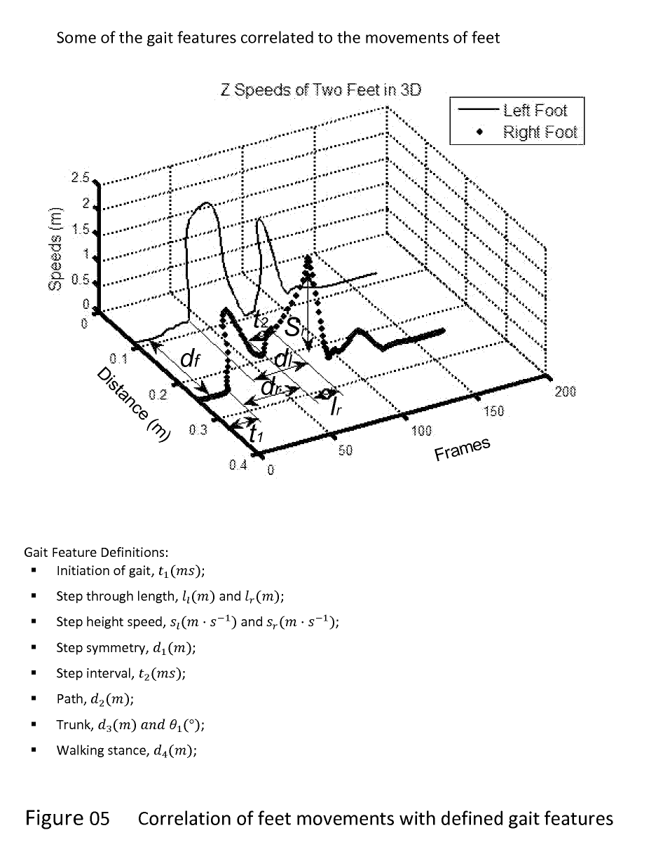

Some of these specific features' values associated with the movements of feet are illustrated in FIG. 5. We define the following features' involved in the 360° turning analysis:
8. Continuity of steps t3 (ms);
9. Steadiness d5 (m)

The classification of normal and abnormal patterns of each gait feature of a subject is performed by setting thresholds for the features' values extracted from the recorded skeleton. To determine the thresholds of the features', data were captured from athletes with potential risk of concussion and a kinesiologist was asked to score the athletes by watching the pre-recorded videos using the software developed for the study. The scores given by the kinesiologist were used as the ground truth for determining the thresholds.

The algorithms were designed using Matlab 2014a for data analysis and later were redesigned and coded in C++. Using Microsoft Visual Studio 2013, a desktop application was designed for performing experimental real-time assessments and further advancement of the designs has created the MAT as a tool for kinesiology professionals, practitioners and clinical testers to use as the new and validated mobility assessment tool. By way of example, the design methods will now be revealed herein.

In this example data were captured from 14 athlete subjects sample group by researchers in the department of Kinesiology at York University. Three athletes had a history of concussions, one had a suspected concussion and the rest were healthy controls. Informed consent was obtained from the participants in accordance with a protocol approved by the Human Participants Review Subcommittee at York University.

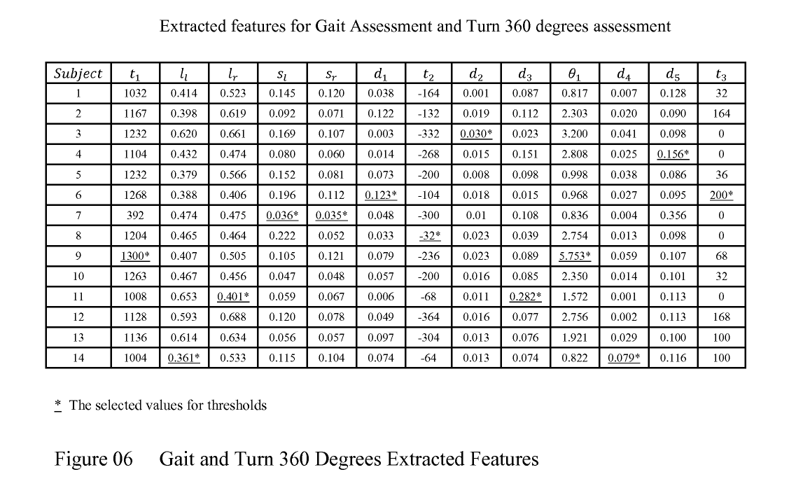

A Kinect V-1 sensor (camera) used was placed 0.84 m above the ground. For gait assessments, the athletes were asked to stand 3.8 m away from the camera, perform a straight line walk towards the camera and stop at 1.8 m away from the camera. For 360° turning assessments, the athletes were asked to stand at a position between 1.8 m and 3.8 m away from the camera and perform a 360° turn. To calibrate the system, the specific features' values to be extracted from the collected data were determined using the developed algorithms, as shown in the table of FIG. 6. From the table, some interesting patterns can be observed. For example, in the range of features' values from all 14 subjects, it took most subjects more than 1000 mil-seconds (ms) to initiate a walk as shown in the second column ti. Ideally, the sample data should cover all normal and abnormal patterns of gait analysis and 360° turning analysis so that it is possible to determine optimal thresholds for each feature.

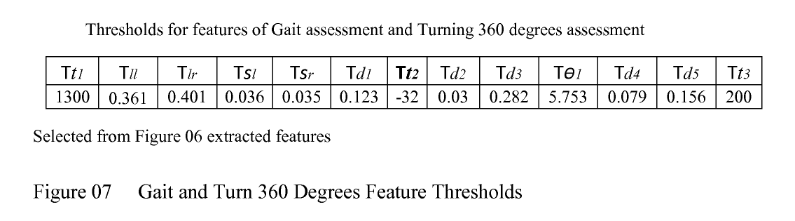

The approach taken to set the thresholds is to consider the 14-subject sample group representative of normal variation. Then, for each feature, the values limit selected is one that will enable all normal participants to pass the automated assessment since all 14 were passed by the kinesiologist's subjective assessment; the limit is normally the value that represents the worst case in a sub-assessment as shown in the table of FIG. 7. For example, in steadiness assessment in 360° turning analysis, the value 0.1564 meter was selected instead of 0.3564 meter because, in the latter case, the subject used arms for balancing which is determined to be abnormal, and which resulted in a longer distance. These features' values thresholds are entered as parameters for the algorithms in the MAT application in this example, for use in experimental real-time assessments of subjects. These parameter settings form the initial baseline for scoring detection of abnormal gaits determined in follow-up clinical studies and subsequently refined to optimize discrimination of normal and abnormal gait for this particular group.

The primary task for any given subject sample group is to build a database that contains as many samples as possible from relevant clinical populations. When the number of samples is large enough and adequately covers normal and abnormal patterns of each gait feature, the accuracy of the determination and segmentation of normal and abnormal gait is improved and new thresholds and more advanced classification algorithms can be determined. It will be clear to anyone with a kinesiology understanding that the MAT methods and algorithms revealed in this patent disclosure, will allow the establishing of databases specialized for clinical populations having particular mobility issues such as related to specific injuries, illnesses, pain, diseases and conditions such as concussion, dementia, chronic pain, Parkinson's, and stroke. The database described by the above example was dealing with male and female subjects in age ranges of 18-25, who are athletic and who have a risk of suffering brain concussions. It will further be clear that due to the objectivity, reliability and reproducibility of the testing mobility of subjects with the MAT system and algorithms, that the results from repeated testing with the MAT of subjects will permit the tracking and monitoring over time, of a subject's particular condition and it's progression of improvement or lack of improvement during treatment being given the subject for that condition. The MAT could become as common and fundamental a medical professional tool as the blood pressure measuring tools found in almost every medical practitioner's office to track and monitor patient's heart and blood pressure cardio vascular condition.

In clinical tests of subjects with the MAT mobility assessment system conducted to date to test and validate the assessment methods and apparatus, it was found that the methods and apparatus were well received by the kinesiology professionals as functional and highly accepted as a unbiased, objective and reproducible tool providing valuable patient mobility information. For the linkage relationships determined between current and previous subject's assessments in evaluating the changes in mobility and mobility impairment and potential existence of concussion as well as and for illness, pain or disease curing, arresting or reversing effects of the illness, pain or disease the MAT was also recognized to be effective.

The system described above has the capability to determine relationships of a subject's present assessments to the subject's previous assessments whereby the expert system can determine and measure the changes in any of the actions and motions of the subject specifically tailored to the subject's individual conditions and health. The expert system not only has databases of information on what are considered normal movements and actions of persons depending on age, sex, health condition and drug use, but also has similar databases specific to the subject being assessed, and thus the expert system can also base-line calibrate its decision-making determinations to what are considered normal movements and actions of the subject being assessed. Determining the relationships to the subject's base-line the expert system can further determine if the present assessment is normal or if it indicates a mobility impairment condition and possible potential existence of injury such as concussion, illness, pain or disease. If the system determines that a mobility impairment condition exists, then the system can determine relationships of the present assessment to previous assessments for this subject to further determine changes in the mobility impairment conditions. Further, if video monitoring in areas where the subject moves about, such as in a residence, home, hospital, playing and sports fields, professional stadium and sports entertainment facilities or natural environments are implemented as the earlier discussion noted, the expert system can determine relationships of these data with which the system can determine the mobility impairment and changes in the subject's mobility in the subject's daily living environment from which the system can determine more comprehensive preventative and remedial practices, health and well-being programs, mobility aids, and monitoring programs for improved quality of life activities, work related activities, monitoring of rehabilitation programs and their success or failure or modifications specific for the subject.

In either real-time or post-recording, the MAT expert system can be the decision-making facility which permits the actual operation of the system and assessment to be done by regular staff of the subject's employer, or clinic, or athletic or sports facilities without the need for highly qualified and expensive professional personnel. This frees up the professional practitioners time by integrating the MAT results into the diagnosis of their patient's mobility and health condition. The apparatus and methods described above can also allow authorized personnel, such as professional physiotherapists, neurologists and concussion specialists to review this new source of mobility assessment data and the determinations made by the MAT system, and integrate this information into their diagnosis of their patients' conditions.

A new and unique embodiment of the expert system is revealed here, that for the first time provides a fully computerized automation implementation of the standard kinesiology mobility test fundamentals of the Tinetti test as a tool for the kinesiology professional which provides consistent, reproducible and reliable testing results across any and all testers. Every subject receives identical computer generated verbal and video instructions, each and every time the subject performs the assessment test. This eliminates inter-tester and intra-tester reliability errors. Instructions are in a variety of selectable languages suited to the subject's requirements. Also, the MAT revealed herein, is designed to save time for both administrators and health care professionals. Assessment results are provided in both gross overall mobility scores and detailed results of the subject's performance of specified movements. Numerical and textual data are provided in readily accessible formats that can quickly and easily be stored and transferred within internal file format frameworks and exported to standardized spreadsheet and word processing formats based on kinesiology practice and setting.

From the above it will be clear the assessment methods and apparatus of the MAT tool described could be applied to many environments, such as, hospitals, private homes, hotels, commercial establishments, doctor's offices, clinics, drugstores, mobility-aids stores, and in the broad sense anywhere people are moving about such as sports and athletic facilities, playing fields, gyms, employment facilities. Also it will be clear to anyone versed in the healthcare field that many different algorithms, algorithm test parameters, action scoring methods and determinations can be implemented, including, mobility impairment algorithms, time derivative determinations and mobility testing, such as those we reveal as incorporated into the computer facility active logic engine neural networks decision determinations methods and apparatus with which we can assess mobility impairment and potential existence of injury, illness, pain or disease, the preventative outcomes and recommendations to reduce further mobility impairment and potential further injury, and for improved quality of life for assessed subjects. Further, it will also be clear that the methods and apparatus of the MAT tool, assessments and recommendations facilitated by the expert system can have application to any subject persons regardless of their age, health, sex, location or activity. Also, it will also be clear that the methods and apparatus, assessments and recommendations facilitated by the expert system can have application to assessment of and the tracking the progression of injury such as concussion, and the effects of treatments and rehabilitation regimes whether trials or long-term such as drugs, physiotherapy, nutrition, exercise, and success or failure of those treatments, and for other conditions such as diseases, illnesses, pains and injuries not limited to only those disclosed herein.

What we claim is:

1. A computerized system for determining the mobility and mobility impairment of a subject performing the 8 physical body movements of: sit-up straight in a chair; stand up from a chair; stand still; stand still with eyes closed; sit down on a chair; walk in a straight-line path; turn 360 degrees walking in a complete circle; turn 360 degrees turning-on-the-spot, said movements following kinesiology practice and protocols from which said movements are extracted 13 features: Initiation of Gait t1 (in milliseconds);
Step Through Length for right foot: lr (in meters); Step Through Length for left foot: ll (in meters); Step Height for right foot: Sr (in meters per second); Step Height for left foot Sl (in meters per second); Step symmetry d1 (in meters); Step Interval t2 (in milliseconds); Path d2 (in meters); Trunk d3 (in meters); Leaning angle θ1 (in degrees);
Walking Stance d4 (in meters); Continuity of steps t3 (in milliseconds); Steadiness d5 (in meters), all of which are representative of said movements from which said features the mobility assessment of said subject are determined, for which said system comprises:
video sensors to observe said movements; video processing interface to determine a video data stream from said sensors frame by frame for example operating at 30 frames per second; video data stream databases for storing said data stream; a computerized active fuzzy logic engine including machine vision and machine learning; video data handling algorithms administered by said engine for accessing and storing said video data streams into said databases; video data handling algorithms administered by said engine for determining a skeleton multi-nodal representation of the said subject's body joints such as head, neck, shoulders, elbows, wrists, trunk, hips, knees, ankles; said skeleton nodal representation determined from said video data stream frame by frame determines a skeleton multi-nodal video data stream; skeleton nodal video data stream databases;
skeleton nodal data handling algorithms administered by said data engine for accessing and storing said skeleton nodal data streams into said skeleton nodal video data databases; further skeleton nodal data stream algorithms administered by said engine to said skeleton nodal data streams and to said skeleton nodal data in said skeleton nodal databases by which administering said further algorithms to the said skeleton nodal data, the said 13 features can be extracted from said skeleton nodal data stream and from said stored skeleton nodal data streams.

2. A system according to claim 1 including further data algorithms administered by said engine for data storage functions and creating databases for storing the said video data stream data and for storing the said skeleton nodal data stream and for storing the said extracted features and for adding and storing maximum and minimum value ranges for each said feature.

3. A system according to claim 2 including further data algorithms administered by said engine for data storage functions and creating databases including a database for entry and storage of results of a kinesiology specialist's subjective manual scoring of a kinesiology standard mobility test for the said movements of the said subject, which scoring results can be used as an independent baseline assessment for the subject's features value ranges permitting said algorithms to adjust the said ranges such that the computerized mobility and mobility impairment assessment determined from said features is in agreement with the said baseline.

4. A system according to claim 3 including further feature algorithms administered by said engine to said extracted features of each of a group of subject's to determine the range of values within said group for each feature by which said algorithms determine the maximum and minimum range for each feature as representative values ranges for said group.

5. A system according to claim 1 including data storage functions and databases for storing the said video data stream data and for storing the said skeleton nodal data stream and for storing the said extracted features and for storing those features and known norms of said features for said subject determined from administration by said active logic engine of said mobility and mobility impairment algorithms to the contents of the said video data stream and skeleton nodal data stream stored in said databases.

6. A method of administration of algorithms for determining mobility and mobility impairment of a subject comprising: the steps of recording video data stream from video sensors observing the movements of said subject; determination of a skeleton nodal data stream of a subject performing 8 body movements including: sit still in a chair; arise up from a chair; stand still; stand still with eyes closed; sit down on a chair; walk in a straight path; walk turning 360 degrees in a circle; walk turning 360 degree on-the-spot, movements of said subject; and further comprising administration by a computerized fuzzy logic engine of fuzzy logic computer algorithms to said video data stream for determination and recording of a skeleton nodal video data stream representation of said subject's body performing said movements; and comprising administration of fuzzy logic computer algorithms to said skeleton nodal data stream for determining 13 extracted features of said movements and storing of said features to a features database;
administration of further fuzzy logic algorithms to record said video data and said video skeleton nodal data as video data streams to databases and recording of said features to features databases and administration of fuzzy logic algorithms for said 8 movements determining the condition of said subject for abnormalities and impairments of such movement, and for determining relationships of said abnormalities and impairments to known norms, and for determining whether said abnormalities and impairments are within a known norms.

7. A method according to claim 6 wherein said 13 extracted features are determined by administration of said algorithms to said skeleton nodal video data to measure the 13 features including: 1. Initiation of Gait t1 (in milliseconds): time consumed between computerized voice instruction “begin” and start of a walk by a subject; 2. Step Through Length for right foot: lr (in meters) and 3. Step Through Length for left foot: ll (in meters): for which from said feature 2. and said feature 3, the mean distance between the ankles of two feet when both of them touch the ground during a walk is determined; 4. Step Height for right foot: Sr (in meters per second) and 5. Step Height for left foot Sl (in meters per second): for which from said feature 4. and said feature 5. the mean speed of a moving foot in the vertical direction is determined; 6. Step symmetry dl (in meters): the mean difference between the Step Length determined by said algorithms for left foot dl (in meters): length of left foot step from step-start at heel lift-up to step-stop at heel put-down and the Step Length determined by said algorithms for right foot dr (in meters):
length of right foot step from step-start at heel lift-up to step-stop at heel put-down; 7. Step Interval t2 (in milliseconds): time consumed between end of a foot step (right or left) and a start of new foot step (right or left); 8. Path d2 (in meters): the mean value of the perpendicular distances of the positions of the center of the trunk with respect to the straight line path movement of the subject walking from a start position in a direction Z directly towards the sensors; 9. Trunk d3 (in meters): the maximum difference between the distance of the resting positions of the subject's wrists and the distance of the wrists during the subject walking; 10. Leaning angle θ1 (in degrees): the leaning angle of the subject's trunk in the coronal plane; 11. Walking Stance d4 (in meters): the difference between the mean distance between the left and right feet in the X direction orthogonal to the vertical Y and the path Z directions; 12. Continuity of steps t3 (in milliseconds): the maximum time consumed in the longest pause between steps when the subject is turning 360 degrees; 13. Steadiness d5 (in meters):the maximum difference between the distances of the resting position of the subjects wrists and the distance between the wrists during the subject walking in a 360 degree turn in a circle or turn on-the-spot of those movements.

8. A method according to claim 6 wherein administration by a computerized fuzzy logic engine of features fuzzy logic computer algorithms determines the maximum and minimum range of values among all subjects of a group of subjects, for each of the extracted features and stores said range values in a range value database representative of the group as a whole.

9. A method according to claim 8 wherein members of a said group are selected specifically such that each subject of said group has similar conditions of mobility and mobility impairment related to such as similar injury, pain, illness, disease, brain concussion, from which administration to the group's feature values range for each extracted feature by said computerized fuzzy logic engine of further features fuzzy logic computer algorithms determines a feature values range baseline for each of the representative for the said conditions and stores that feature values range in a ranges database.

10. A method according to claim 9 wherein said computerized fuzzy logic engine applies further features fuzzy logic computer algorithms to determine if a given subject's features values most closely match and fall within the ranges of a said specific selected groups' features values ranges stored in said ranges database containing a collection of many said groups with varying degrees of mobility and mobility impairments, by which determination the said subject's condition of mobility and mobility impairments are assessed to be those of the most closely said matched group.

11. A method according to claim 10 wherein said range of values for each feature for a said selected group are determined by administration by said engine of further fuzzy logic computer algorithms at a specific stage of the said impairment conditions of said selected group such that several groups selected, each representative for an give stage of said impairments determines a set of groups representative of many stages of said impairment.

12. A method according to claim 11 wherein said computerized fuzzy logic engine applies further features fuzzy logic computer algorithms to determine the features assess for a specific subject and determines from the said set of groups that group to which the said subject's features most closely lay and match, by which said engine applies said algorithms to repeated feature assessments and matching over a period of time to determine and track in time the assessments of the said features of said subject from which said algorithms determine potential recovery, success of treatments, and possibly decay of the subject's mobility and mobility impairment.
